(19) United States
(12) Patent Application Publication
(10) Pub. No.: US 2023/0083509 A1
(43) Pub. Date: Mar. 16, 2023

(54) POLYACRYLATE-FREE COSMETIC PREPARATION

(71) Applicant: BEIERSDORF AG, Hamburg (DE)

(72) Inventors: David SCHLENKER, Hamburg (DE); Stephanie VON DER FECHT, Wedel (DE); Heike VOLBRICH, Hamburg (DE); Sarah SPROCK, Hamburg (DE)

(21) Appl. No.: 17/904,039

(22) PCT Filed: Jan. 14, 2021
(86) PCT No.: PCT/EP2021/050661

§ 371 Date: Aug. 11, 2022

(30) Foreign Application Priority Data
Feb. 13, 2020 (DE) 10 2020 201 799.9

(51) Int. Cl. A61K 8/73; A61K 8/35; A61K 8/41; A61K 8/49; A61K 8/92; A61K 8/06; A61Q 17/04

(52) U.S. Cl. CPC A61K 8/731; A61K 8/73; A61K 8/35; A61K 8/415; A61K 8/4966; A61K 8/922; A61K 8/062; A61Q 17/04; A61K 2800/30; A61K 2800/48

(57) ABSTRACT

Cosmetic preparation containing a) hydroxypropyl methyl cellulose and b) xanthan gum, the preparation being free from polyacrylates, carbomers and polyvinylpyrrolidones.

The present invention relates to a cosmetic preparation comprising hydroxypropyl methylcellulose and xanthan gum, the preparation being free from polyacrylates, carbomers and polyvinyl-pyrrolidones, and to methods and uses of hydroxypropyl methylcellulose and xanthan gum in polyacrylate-free cosmetic preparations.

There has been a continuing trend away from pale complexion towards “healthy, athletic tanned skin” for many years. To achieve this, people expose their skin to sunlight, since this causes pigment formation in the sense of melanin formation. However, the ultraviolet radiation of sunlight also has a harmful effect on skin. In addition to acute damage (sunburn), long-term damage such as an increased risk of developing skin cancer occurs on excessive irradiation with light from the UVB range (wavelength: 280-320 nm). Excessive exposure to UVB and UVA radiation (wavelength: 320-400 nm) additionally leads to a weakening of the elastic and collagen fibers in connective tissue. This leads to numerous phototoxic and photoallergic reactions and results in premature aging of the skin.

To protect the skin, a number of photoprotective filter substances have accordingly been developed that may be used in cosmetic preparations. These UVA and UVB filters are in most industrialized countries collated in the form of positive lists such as Annex 7 of the Kosmetikverordnung [German Cosmetics Ordinance]

The plethora of commercially available sunscreens must not however obscure the fact that these prior art preparations have a number of disadvantages.

A widespread form of use of cosmetic sunscreens is in the context of water sports. Sunscreens are often used in connection with beach holidays, visits to swimming pools, when bathing, surfing, diving, paddling or other activities where human skin comes into contact with water.

For all these activities, it is important for comprehensive UV protection that the sunscreen has a certain water resistance and is not immediately rinsed off the skin on contact with the water.

To increase the water resistance of cosmetic sunscreens, so-called film formers are generally added to these preparations. The film formers are usually polymeric compounds based on polyacrylates or polyvinylpyrrolidones, which form a protective film on the skin which is intended to prevent/delay the UV filters from being washed off the skin.

Polyacrylates are also used as a substitute for low-molecular weight emulsifiers. Their emulsifying effect is based on the formation of a gel network in the preparation, which stabilizes the oil droplets in the aqueous environment and prevents them from coalescing.

The use of such polymers is viewed increasingly critically by the wider public. Concerns are often voiced in the public debate that such polymers, analogous to so-called “microplastics”, could pose a burden on the environment. Whether such concerns are justified and scientifically valid can be left open in the context of the present disclosure. The fact is, however, that there is increasing interest among cosmetic manufacturers and consumers in developing alternative techniques to ensure the water resistance and emulsion stability of sunscreens and other cosmetics. The preparations should be “microplastic-free” according to the understanding of the consumer.

It was therefore the object of the present invention to develop a sunscreen which is as waterproof as possible and in which the use of polymeric film formers (particularly those based on polyacrylates and polyvinylpyrrolidones) can largely be dispensed with. It was also the object of the present invention to develop a cosmetic preparation in which the use of synthetic polymers and particularly those based on polyacrylates can be dispensed with.

Surprisingly, the object is achieved by a cosmetic preparation comprising

a) hydroxypropyl methylcellulose and
b) xanthan gum,
wherein the preparation is free from polyacrylates, carbomers and polyvinylpyrrolidones.

Surprisingly, the object is also achieved by a method for regulating the viscosity of polyacrylate-free cosmetic preparations, characterized in that a mixture of hydroxypropyl methylcellulose and xanthan gum is added to the preparation.

The object is surprisingly achieved not least by the use of a mixture of hydroxypropyl methylcellulose and xanthan gum as a substitute for polyacrylates in polyacrylate-free cosmetic preparations.

Preparations with the combination of ingredients according to the invention have a higher temperature and storage stability compared with other celluloses and gums. While phase separation (oil separation) was observed after a longer storage time or increased storage temperature when using other natural thickeners, such as hydroxypropyl starch phosphate, microcrystalline cellulose or gellan gum, the combination of substances according to the invention proved to be surprisingly stable.

In the context of the present disclosure, the phrases “according to the invention”, “advantageous according to the invention” etc. always refer to the preparation according to the invention, the use according to the invention and the method according to the invention, unless otherwise described in the individual case.

According to the invention, UV filters are all substances that are listed as UV filters in Annex VI of Regulation (EU) No. 866/2014 and Regulation No. 1223/2009 of the EU Commission.

According to the invention, the hydroxypropyl methylcellulose used is advantageously a compound having an average molecular weight of 80 000 to 90 000 g/mol. According to the invention, a 2% aqueous solution of hydroxypropyl cellulose preferably has a viscosity of 400 cPs at 20° C. For example, Methocel E4M Premium from Dow Chemical or Tylose E707002 from SE Tylose GmbH & Co. KG may be used advantageously according to the invention.

According to the invention, the xanthan gum used can advantageously be a compound having a viscosity, at 1% in a 1% KCl solution, of 1200-1600 mPa*s (cP) under standard conditions at 60 rpm. Advantageously according to the invention, a 0.25% solution in tap water has a viscosity of 600-1200 mPa*s (cP) at 3 rpm under standard conditions. Advantageously according to the invention, the xanthan gum according to the invention has a particle size of 200 mesh in the dry state. For example, Keltrol CG-F V from CP Kelco or Xanthan Gum food grade, Type FF and Type FFPC from Jungbunzlauer may be used advantageously in accordance with the invention.

Embodiments of the present invention that are advantageous according to the invention are characterized in that the preparation comprises one or more UV filters selected from the group of compounds comprising 4-(tert-butyl)-4′-methoxydibenzoylmethane (INCI Butyl Methoxydibenzoylmethane), hexyl (2-[4-(diethylamino)-2-hydroxybenzoyl]benzoate (INCI: Diethylamino Hydroxybenzoyl Hexyl Benzoate) and 2,4-bis{[4-(2-ethylhexyloxy)-2-hydroxy]phenyl}-6-(4-methoxyphenyl)-1,3,5-triazine (INCI Bis-Ethylhexyloxyphenol Methoxyphenyl Triazine).

If the preparation comprises 4-(tert-butyl)-4′-methoxydibenzoylmethane (INCI Butyl Methoxydibenzoylmethane), the advantageous according to the invention is 1 to 5% by weight, based on the total weight of the preparation.

If the preparation comprises hexyl (2-[4-(diethylamino)-2-hydroxybenzoyl]benzoate (INCI: Diethylamino Hydroxybenzoyl Hexyl Benzoate), the advantageous use concentration according to the invention is 1 to 9% by weight, based on the total weight of the preparation.

If the preparation comprises 2,4-bis{[4-(2-ethylhexyloxy)-2-hydroxy]phenyl}-6-(4-methoxyphenyl)-1,3,5-triazine (INCI Bis-Ethylhexyloxyphenol Methoxyphenyl Triazine), the advantageous use concentration according to the invention is 0.5 to 4.5% by weight, based on the total weight of the preparation.

Furthermore, advantageous embodiments according to the invention are characterized in that they comprise one or more UV filters selected from the group of the compounds 2-phenylbenzimidazole-5-sulfonic acid salts; phenylene-1,4-bis(2-benzimidazyl)-3,3′-5,5′-tetrasulfonic acid salts; 1,4-di(2-oxo-10-sulfo-3-bornylidenemethyl) benzene and salts thereof; 4-(2-oxo-3-bornylidenemethyl) benzenesulfonic acid salts; 2-methyl-5-(2-oxo-3-bornylidenemethyl)sulfonic acid salts; 2,2′-methylenebis(6-(2H-benzotriazol-2-yl)-4-(1,1,3,3-tetramethylbutyl)phenol); 2-(2H-benzotriazol-2-yl)-4-methyl-6-[2-methyl-3-[1,3,3,3-tetramethyl-1-[trimethylsilyl)oxy]disiloxanyl]propyl]phenol; ethylhexyl salicylate; terephthalidenedicamphorsulfonic acid salts; 2-ethylhexyl 2-cyano-3,3-diphenylacrylate; amyl 4-(dimethylamino) benzoate; di(2-ethylhexyl) 4-methoxybenzalmalonate; isoamyl 4-methoxycinnamate; 2-hydroxy-4-methoxy-4′-methylbenzophenone; 2,2′-dihydroxy-4-methoxybenzophenone; homomenthyl salicylate; 2-ethylhexyl 2-hydroxybenzoate; dimethicodiethylbenzalmalonate; 3-(4-(2,2-bis ethoxycarbonylvinyl)phenoxy)propenyl)methoxysiloxane/dimethylsiloxane-copolymer; dioctylbutylamidotriazone (INCI: Diethylhexyl Butamidotriazone); 2,4-bis[5-1(dimethylpropyl)benzoxazol-2-yl-(4-phenyl)imino]-6-(2-ethylhexyl)imino-1,3,5-triazine with (CAS No. 288254-16-0); tris(2-ethylhexyl) 4,4′,4″-(1,3,5-triazine-2,4,6-triyltriimino)trisbenzoate (also: 2,4,6-tris[anilino-(p-carbo-2′-ethyl-1′-hexyloxy)]-1,3,5-triazine (INCI: Ethylhexyl Triazone); 2,4,6-tribiphenyl-4-yl-1,3,5-triazine; merocyanine; piperazine derivatives; titanium dioxide; zinc oxide.

Preference is given in accordance with the invention in particular to the use of phenylbenzimidazole-5-sulfonic acid salts, ethylhexyl salicylate, homomenthyl salicylate, 2-ethylhexyl 2-cyano-3,3-diphenyl acrylate, dioctylbutylamidotriazone (INCI: Diethylhexyl Butamidotriazone), tris(2-ethylhexyl) 4,4′,4″-(1,3,5-triazine-2,4,6-triyltriimino)trisbenzoate (INCI: Ethylhexyl Triazone), titanium dioxide. Particularly preferred according to the invention are the UV filters phenylbenzimidazole-5-sulfonic acid salts, ethylhexyl salicylate, dioctylbutylamidotriazone (INCI: Diethylhexyl Butamidotriazone), tris(2-ethylhexyl) 4,4′,4″-(1,3,5-triazine-2,4,6-triyltriimino) trisbenzoate (INCI: Ethylhexyl Triazone).

The preferred use concentration according to the invention of phenylbenzimidazole-5-sulfonic acid salts is from 0.5 to 4% by weight, based on the total weight of the preparation; the preferred use concentration of ethylhexyl salicylate is from 0.5 to 5% by weight, based on the total weight.

It is advantageous in the context of the present invention if the preparation comprises one or more UV filters a) at a total concentration of 1 to 30% by weight, based on the total weight of the preparation.

It is advantageous according to the invention if the preparation comprises hydrogenated rapeseed oil (INCI: Hydrogenated Rapeseed Oil). This may be purchased, for example, under the trade name Dermofeel Viscolid pof from Evonik Nutrition & Care GmbH or Dr. Straetmans GmbH.

It is advantageous according to the invention if the preparation comprises hydrogenated rapeseed oil (INCI: Hydrogenated Rapeseed Oil) at a concentration of 0.5 to 3.0% by weight, based on the total weight of the preparation.

Advantageous embodiments of the present invention are also characterized in that the preparation is free from 3-(4-methylbenzylidene)camphor, 2-hydroxy-4-methoxybenzophenone (INCI: Oxybenzone), 2-ethylhexyl 4-methoxycinnamate (INCI Octyl Methoxycinnamate), parabens (particularly propyl and butyl paraben), methylisothiazolinone, chloromethylisothiazolinone and DMDM hydantoin, polyethylene glycol ethers or polyethylene glycol esters.

It is advantageous according to the invention if the preparation comprises hydroxypropyl methylcellulose at a concentration of 0.05 to 0.5% by weight and xanthan gum at a concentration of 0.1 to 1% by weight, based in each case on the total weight of the preparation.

It is preferred according to the invention if the preparation comprises hydroxypropyl methylcellulose at a concentration of 0.1 to 0.5% by weight and xanthan gum at a concentration of 0.3 to 0.7% by weight, based in each case on the total weight of the preparation.

It is also advantageous in the context of the present invention if the ratio by weight of hydroxypropyl methylcellulose to xanthan gum in the preparation is from 1:2 to 2:7.

It is advantageous according to the invention if the preparation is in the form of an O/W emulsion or hydrodispersion.

If the preparation according to the invention is in the form of an O/W emulsion, it is advantageously characterized according to the invention in that the preparation comprises one or more O/W emulsifiers selected from the group of the compounds glyceryl stearate citrate, glyceryl stearate (self-emulsifying), stearic acid, stearate salts, polyglyceryl-3 methylglycose distearate, sodium cetearyl sulfate, potassium cetyl phosphate, polyglyceryl-10 stearate, sodium stearoyl glutamate.

It is advantageous in accordance with the invention if the preparation comprises ethanol. It is preferable in accordance with the invention to use ethanol at a concentration of 2 to 10% by weight based on the total weight of the preparation.

Advantageous embodiments in the context of the present invention are characterized in that the preparation comprises ethylhexylglycerin and/or 4-hydroxyacetophenone.

It is also advantageous according to the invention if the preparation comprises one or more alkanediols from the group of the compounds pentane-1,2-diol, hexane-1,2-diol, octane-1,2-diol, decane-1,2-diol, 2-methylpropane1,3-diol.

Embodiments of the present invention that are advantageous in accordance with the invention are characterized in that the preparation comprises one or more compounds selected from the group of the compounds alpha-lipoic acid, folic acid, phytoene, D-biotin, coenzyme Q10, alpha-glucosylrutin, carnitine, carnosine, natural and/or synthetic isoflavonoids, flavonoids, creatine, creatinine, taurine, β-alanine, panthenol, magnolol, honokiol, tocopheryl acetate, dihydroxyacetone, 8-hexadecene-1,16-dicarboxylic acid, thiamidol, glycerylglucose, (2-hydroxyethyl)urea, vitamin E and derivatives thereof, hyaluronic acid and/or salts thereof, and/or licochalcone A.

The preparation according to the invention may advantageously contain moisturizers. Moisturizers are substances or mixtures of substances that give cosmetic preparations the property, when applied or spread on the surface of the skin, of reducing the loss of moisture from the stratum corneum (also known as transepidermal water loss (TEWL)) and/or of having a beneficial effect on the hydration of the stratum corneum.

Advantageous moisturizers according to the present invention are, for example, glycerin, lactic acid and/or lactates, in particular sodium lactate, butylene glycol, propylene glycol, biosaccharide gum-1, glycine soya, ethylhexyloxyglycerin, pyrrolidone carboxylic acid and urea. Furthermore, it is particularly advantageous to use polymeric moisturizers from the group of polysaccharides which are soluble in water and/or swellable in water and/or gellable with the aid of water. Particularly advantageous, for example, are hyaluronic acid, chitosan and/or a fucose-rich polysaccharide, which is listed in Chemical Abstracts under the registration number 178463-23-5 and is obtainable, for example under the name Fucogel®1000 from SOLABIA S.A. It is also possible to use moisturizers advantageously as anti-wrinkle agents, for protection against skin changes which occur during ageing of the skin for example.

The cosmetic preparations according to the invention may also advantageously comprise fillers, although not mandatory, which further improve, for example, the sensory and cosmetic properties of the formulations and which generate or enhance, for example, a velvety or silky skin feel. Advantageous fillers in the context of the present invention are starch and starch derivatives (such as tapioca starch, distarch phosphate, aluminum or sodium starch octenyl succinate and the like), pigments, which have principally neither UV filter nor staining effects (e.g., boron nitride etc.) and/or Aerosils® (CAS No. 7631-86-9) and/or talc.

It is preferred in accordance with the invention if the preparation according to the invention comprises tapioca starch and/or distarch phosphate.

Embodiments of the present invention that are advantageous in accordance with the invention are characterized in that the preparation comprises one or more oils selected from the group of the compounds Butylene Glycol Dicaprylate/Dicaprate, Phenethyl Benzoate, C12-15 Alkyl Benzoate, Dibutyl Adipate, Diisopropyl Sebacate, Dicaprylyl Carbonate, Di-C12-13 Alkyl Tartrate, Butyloctyl Salicylate, Diethylhexyl Syringylidenemalonate, Hydrogenated Castor Oil Dimerate, Triheptanoin, C12-13 Alkyl Lactate, C16-17 Alkyl Benzoate, Propylheptyl Caprylate, Caprylic/Capric Triglyceride, Diethylhexyl 2,6-Naphthalate, Octyldodecanol, Caprylic/Capric Triglyceride, Ethylhexyl Cocoate.

In this case it is preferred in accordance with the invention if the preparation comprises dibutyl adipate, dicaprylyl carbonate, butylene glycol dicaprylate/dicaprate and/or C12-C15 alkyl benzoate.

The water phase of the preparations according to the invention may advantageously comprise customary cosmetic auxiliaries, such as alcohols, in particular those having a low number of carbon atoms, preferably ethanol and/or isopropanol or polyols of low carbon number and ethers thereof, preferably propylene glycol, glycerin, electrolytes, self-tanners, etc.

It is further advantageous in the context of the invention if the preparation according to the invention comprises one or more perfumes selected from the group of the compounds limonene, citral, linalool, alpha-isomethyl ionone, geraniol, citronellol, 2-isobutyl-4-hydroxy methyltetrahydropyran, 2-tert-pentylcyclohexyl acetate, 3-methyl-5-phenyl-1-pentanol, 7-acetyl-1,1,3,4,4,6-hexamethyltetralin, adipic acid diesters, alpha-amylcinnamaldehyde, alpha-methyl ionone, amyl C butylphenylmethylpropionalcinnamal, amyl salicylate, amylcinnamyl alcohol, anise alcohol, benzoin, benzyl alcohol, benzyl benzoate, benzyl cinnamate, benzyl salicylate, bergamot oil, bitter orange oil, butylphenyl methylpropional, cardamom oil, cedrol, cinnamal, cinnamyl alcohol, citronellyl methylcrotonate, lemon oil, coumarin, diethyl succinate, ethyl linalool, eugenol, Evernia furfuracea extract, Evernia prunastri extract, farnesol, guaiac wood oil, hexylcinnamal, hexyl salicylate, hydroxycitronellal, lavender oil, limonene oil, linayl acetate, mandarin oil, menthyl PCA, methylheptenone, nutmeg oil, rosemary oil, sweet orange oil, terpineol, tonka bean oil, triethyl citrate and/or vanillin.

In addition, the preparations according to the invention may comprise ingredients customary for cosmetic sunscreens at customary use concentrations.

### EXAMPLES

The examples below are intended to illustrate the present invention without limiting it. Unless otherwise stated, all quantitative data, fractions, and percentages are based on the weight and the total amount or on the total weight of the preparations.

| | |
|---|---|
| Aqua | 60.95 |
| Tocopheryl Acetate | 0.06 |
| Aqua + Trisodium EDTA | 0.50 |
| Phenoxyethanol | 0.40 |
| Dimethicone | 0.90 |
| C12-15 Alkyl Benzoate | 5.00 |
| Butyl Methoxydibenzoylmethane | 4.75 |
| Phenylbenzimidazole Sulfonic Acid | 0.50 |
| Behenyl Alcohol | 1.20 |
| Glycerin | 1.00 |
| Aqua + Sodium Hydroxide | 0.17 |
| Alcohol Denat. + Aqua | 7.50 |
| Ethylhexyl Salicylate | 2.00 |
| Xanthan Gum | 0.30 |
| Hydroxypropyl Methylcellulose | 0.20 |
| Ethylhexyl Triazone | 1.50 |
| Silica Dimethyl Silylate | 0.50 |
| Stearyl Alcohol | 1.20 |
| Bis-Ethylhexyloxyphenol Methoxyphenyl | 2.50 |
| Triazine | |
| Butylene Glycol Dicaprylate/Dicaprate | 2.00 |
| Glycyrrhiza Inflata Root Extract | 0.03 |
| Diethylamino Hydroxybenzoyl Hexyl | 1.00 |
| Benzoate | |
| Sodium Stearoyl Glutamate + Sodium | 0.25 |
| Chloride | |
| Titanium Dioxide (nano) + Silica + | 3.00 |
| Dimethicone | |
| Hydroxyacetophenone | 0.20 |
| Hydrogenated Rapeseed Oil | 2.00 |
| Perfume | 0.40 |
| Total: | 100.00 |

| | |
|---|---|
| Isopropyl Palmitate | 6.00 |
| Aqua | 60.70 |
| Tocopheryl Acetate | 0.06 |
| Aqua + Trisodium EDTA | 0.50 |
| Phenoxyethanol | 0.40 |
| Dimethicone | 0.90 |
| C12-15 Alkyl Benzoate | 5.00 |
| Butyl Methoxydibenzoylmethane | 2.40 |
| Phenylbenzimidazole Sulfonic Acid | 0.50 |
| Behenyl Alcohol | 1.50 |
| Glycerin | 1.00 |
| Aqua + Sodium Hydroxide | 0.17 |
| Alcohol Denat. + Aqua | 7.50 |
| Ethylhexyl Salicylate | 2.00 |
| Xanthan Gum | 0.30 |
| Hydroxypropyl Methylcellulose | 0.20 |
| Ethylhexyl Triazone | 0.50 |
| Silica Dimethyl Silylate | 0.50 |
| Stearyl Alcohol | 1.50 |
| Bis-Ethylhexyloxyphenol Methoxyphenyl | 2.00 |
| Triazine | |
| Butylene Glycol Dicaprylate/Dicaprate | 2.00 |
| Glycyrrhiza Inflata Root Extract | 0.03 |
| Diethylamino Hydroxybenzoyl Hexyl | 0.50 |
| Benzoate | |
| Sodium Stearoyl Glutamate + Sodium | 0.25 |
| Chloride | |
| Titanium Dioxide (nano) + Silica + | 2.00 |
| Dimethicone | |
| Hydroxyacetophenone | 0.20 |
| Hydrogenated Rapeseed Oil | 1.00 |
| Perfume | 0.40 |
| Total: | 100.00 |

1.-11. (canceled)

12. A cosmetic preparation, wherein the preparation comprises
(a) hydroxypropyl methylcellulose and
(b) xanthan gum,
the preparation being free from polyacrylates, carbomers and polyvinylpyrrolidones.

13. The preparation of claim 12, wherein the preparation further comprises one or more UV filters selected from 4-(tert-butyl)-4′-methoxydibenzoylmethane (INCI Butyl Methoxydibenzoylmethane), hexyl (2-[4-(diethylamino)-2-hydroxybenzoyl]benzoate (INCI: Diethylamino Hydroxybenzoyl Hexyl Benzoate), 2,4-bis{[4-(2-ethylhexyloxy)-2-hydroxy]phenyl}-6-(4-methoxyphenyl)- 1,3,5-triazine (INCI Bis-Ethylhexyloxyphenol Methoxyphenyl Triazine).

14. The preparation of claim 12, wherein the preparation further comprises hydrogenated rapeseed oil (INCI: Hydrogenated Rapeseed Oil).

15. The preparation of claim 12, wherein the preparation is free from 3-(4-methylbenzylidene)camphor, 2-hydroxy-4-methoxybenzophenone (INCI: Oxybenzone), 2- ethylhexyl 4-methoxycinnamate (INCI Octyl Methoxycinnamate), parabens, methylisothiazolinone, chloromethylisothiazolinone, DMDM hydantoin, polyethylene glycol ethers, polyethylene glycol esters.

16. The preparation of claim 12, wherein the preparation comprises from 0.05% to 0.5% by weight hydroxypropyl methylcellulose and from 0.1% to 1% by weight xanthan gum, based in each case on a total weight of the preparation.

17. The preparation of claim 12, wherein the preparation comprises from 0.1% to 0.5% by weight hydroxypropyl methylcellulose and from 0.3% to 0.7% by weight xanthan gum, based in each case on a total weight of the preparation.

18. The preparation of claim 12, wherein a ratio by weight of hydroxypropyl methylcellulose to xanthan gum in the preparation is from 1:2 to 2:7.

19. The preparation of claim 17, wherein a ratio by weight of hydroxypropyl methylcellulose to xanthan gum in the preparation is from 1:2 to 2:7.

20. The preparation of claim 14, wherein the preparation comprises from 0.5% to 3% by weight of hydrogenated rapeseed oil, based on a total weight of the preparation.

21. The preparation of claim 16, wherein the preparation comprises from 0.5% to 3% by weight of hydrogenated rapeseed oil, based on a total weight of the preparation.

22. The preparation of claim 19, wherein the preparation comprises from 0.5% to 3% by weight of hydrogenated rapeseed oil, based on a total weight of the preparation.

23. The preparation of claim 12, wherein the preparation further comprises hydroxyacetophenone.

24. The preparation of claim 14, wherein the preparation further comprises hydroxyacetophenone.

25. The preparation of claim 12, wherein (a) has an average molecular weight of from 80,000 to 90,000 g/mol.

26. The preparation of claim 12, wherein a 1% solution of (b) in 1% KCl solution has a viscosity of 1,200-1,600 cP under standard conditions at 60 rpm.

27. The preparation of claim 12, wherein the preparation further comprises from 1% to 30% by weight of one or more UV filters, based on a total weight of the preparation.

28. The preparation of claim 12, wherein the preparation is present in the form of an O/W emulsion.

29. The preparation of claim 12, wherein the preparation is present in the form of a hydrodispersion.

30. A method of reducing the viscosity of a polyacrylate-free cosmetic preparation, wherein the method comprises incorporating hydroxypropyl methylcellulose and xanthan gum in the preparation.

31. A method of replacing polyacrylate in a cosmetic preparation, wherein the method comprises incorporating methylcellulose and xanthan gum as substitute for polyacrylate in the preparation.
